(19) United States
(12) Patent Application Publication
(10) Pub. No.: US 2010/0113878 A1
(43) Pub. Date: May 6, 2010

(54) Endoscopic multi-purpose treatment hood and endoscope

(75) Inventor: Tatsuyuki Kawano, Tokyo (JP)

Correspondence Address: OLIFF & BERRIDGE, PLC, P.O. BOX 320850, ALEXANDRIA, VA, 22320-4850, US

(73) Assignee: National University Corporation Tokyo Medical and Dental University, Tokyo (JP)

(21) Appl. No.: 12/223,653

(22) PCT Filed: Feb. 5, 2007
(86) PCT No.: PCT/JP2007/051933

§ 371 Date: Aug. 6, 2008

(30) Foreign Application Priority Data
Feb. 9, 2006 (JP) 2006-032659

(51) Int. Cl. A61B 1/00

(52) U.S. Cl. 600/123

(57) ABSTRACT

On the periphery of a silicon tube 12, a plurality of insertion holes 14 extending in the axial direction of the silicon tube 12 for fitting treatment tools such as a precutting knife 30, a puncture needle 32, a hook knife 34, and a high-frequency snare 36 are formed. Accordingly, a high-versatility endoscopic multi-purpose treatment hood which makes easy detailed examinations, complicated procedures, and difficult treatments using an endoscope can be provided.

### TECHNICAL FIELD

The present invention relates to a special endoscopic multi-purpose treatment hood and endoscope which can be used for various endoscopic treatments, and more specifically, to high-versatility endoscopic multi-purpose treatment hood which makes easy detailed examinations, complicated procedures, and difficult treatments using an endoscope, and an endoscope fitted with this.

### BACKGROUND ART

In an endoscopic treatment of superficial esophageal cancer, to utilize less invasiveness and from the perspective regarding diagnostic aspects and treatment reliability as important, for positively introducing endoscopic resection and collective resection in a treatment, EMR (Endoscopic Mucosal Resection) which enables wide-area resection has been employed. Recently, based on the same idea, introduction of ESD (Endoscopic Submucosal Dissection) with higher reliability for wide-area collective resection has been demanded, however, this takes a long time, so that it is only partially employed.

On the other hand, related to the present invention, an endoscopic hood knife has been proposed in Japanese Laid-Open Patent Publication No. 2005-192675 (Patent document 1). It has a cautery knife function or knife function attached to a hood to be fitted to the tip end of an endoscope so that the attached function can be enabled without using a forceps hole. Therefore, manipulations such as resection, dissection, and avulsion in a visually stable field can be enabled by inserting a treatment tool such as grasping forceps into the forceps channel of the endoscope and grasping a lesion or resection plane.

However, the endoscopic hood knife described in Patent document 1 is mainly used for treatment of digestive system cancers, and cannot be used for multiple purposes, and cannot shorten the treatment time.

### DISCLOSURE OF THE INVENTION

The present invention has been made for solving the above problems related to the conventional method. An object of the invention is to provide an endoscopic multi-purpose treatment hood which can be used for multi-purposes and can remarkably shorten a treatment time of, in particular, superficial esophageal cancer, and an endoscope fitted with this hood.

The present invention solves the above-described problem by a tube-like endoscopic multi-purpose treatment hood including a treatment tool fitting means formed on the periphery.

Herein, the treatment tool fitting means can be a plurality of insertion holes extending in the axial direction of the tube.

Alternatively, the treatment tool fitting means can be formed by rolling a plate having a plurality of convex lines so that the convex lines extend in the axial direction on the outer side and covering a tube thereon.

According to the present invention, by the endoscopic multi-purpose treatment hood provided with a liquid injecting treatment tool in the treatment fitting means, a lesion can be cut in a floating state.

The present invention further provides an endoscope fitted with the hood.

According to the present invention, a means (for example, a plurality of insertion holes or slits extending in the axial direction of a tube) for fitting various treatment tools is formed on the periphery of a tube-like hood, so that separately from the treatment tool such as grasping forceps inserted in a forceps hole of an endoscope, only necessary treatment tools can be easily fitted as occasion may demand, and for example, various treatments using these in combination become possible, and therefore, treatments which were conventionally performed inevitably separately can be collectively performed and lesions can be collectively resected, so that the treatment time can be shortened.

A treatment tool may be inserted in the endoscope, however, for example, when a physiological salt solution is injected, injection of a physiological salt solution and insertion of a treatment tool cannot be performed concurrently, and the treatment must be switched. However, a physiological salt solution spreads immediately, so that in the case of switching with the treatment tool after the physiological salt solution is injected, the lesion cannot be cut in a floating state. On the other hand, according to the multi-purpose treatment hood of the present invention, the lesion can be floated by injecting a physiological salt solution and then cut immediately, and the treatment becomes easy.

Different from a treatment tool inserted through the endoscope, the hood can be rotated.

Further, in the case where a treatment tool is inserted through an endoscope, there is a danger that the treatment tool goes aslant and perforates through the wall surface of a living body, however, according to the present invention, the treatment tool goes only parallel to the hood, so that there is no possibility that the treatment tool damages the wall surface of the living body.

Due to the simple structure of the hood, the hood can be manufactured by using soft silicon which is living-body-friendly, and its shape is tubular and advantageous, so that the hood has excellent practicability.

### BRIEF DESCRIPTION OF THE DRAWINGS

FIG. 1 is a perspective view showing a construction of a first embodiment of an endoscopic multi-purpose treatment hood according to the present invention;

FIG. 2 is a perspective view showing a state where the first embodiment is fitted to the tip end of an endoscope;

FIG. 3 is a perspective view showing a construction example of the entire endoscope to which the first embodiment is fitted;

FIG. 4 is a sectional view showing a state of simple-cutting by the first embodiment;

FIG. 5 is a sectional view showing a state of swing-cutting by the same;

FIG. 6 is a sectional view showing a state of pull-swing cutting by the same;

FIG. 7 are perspective views showing a second embodiment of the present invention;

FIG. 8 is a perspective view showing a variation of the second embodiment; and

FIG. 9 are perspective views showing variations of the first and the second embodiment.

FIG. 10 is a perspective view showing a variation of the first embodiment.

### BEST MODE FOR CARRYING OUT THE INVENTION

An embodiment of the present invention will be described with reference to the drawings.

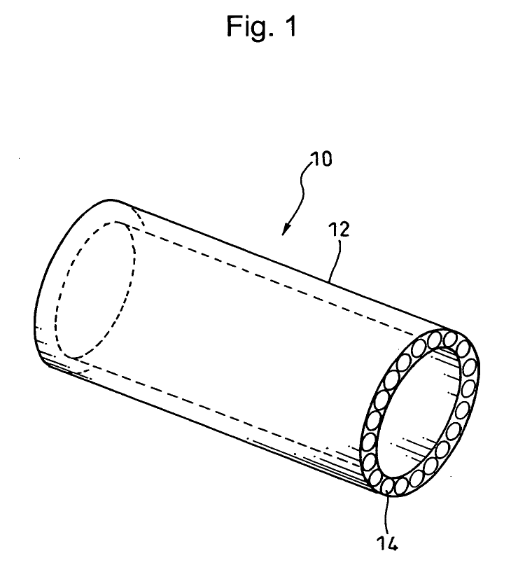

An endoscopic multi-purpose treatment hood (hereinafter, also referred to as hood, simply) 10 of a first embodiment is formed of a cylindrical transparent silicon tube 12 having many insertion holes 14 extending axially arranged in parallel on the periphery for fitting treatment tools as shown in FIG. 1.

The silicon tube 12 has, for example, an entire length of 20 millimeters, an inner diameter of 10 millimeters, and an outer diameter of 12.5 millimeters, and the diameter of the insertion hole 14 can be set to, for example, 1 millimeter.

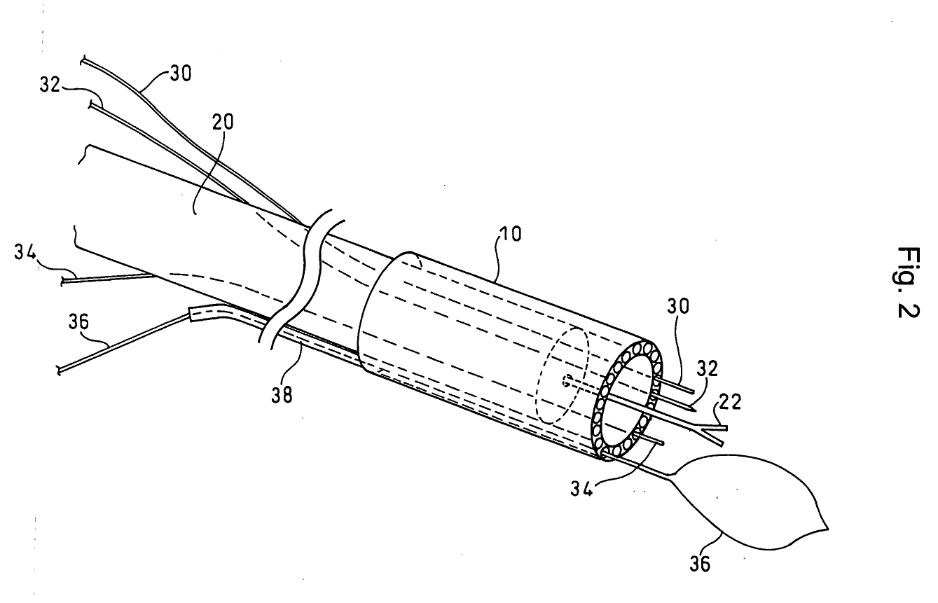

A fitted state of the first embodiment to an endoscope is shown in FIG. 2. This hood 10 is fitted to the tip end of a general-purpose endoscope 20, and through the insertion holes 14, various treatment tools such as a precutting knife 30, a puncture needle 32, a hook knife (registered trademark) 34, a high-frequency snare 36 are inserted. In the figure, the reference numeral 22 denotes grasping forceps inserted through the inside of the endoscope 20, and 38 denotes a treatment tool guiding line.

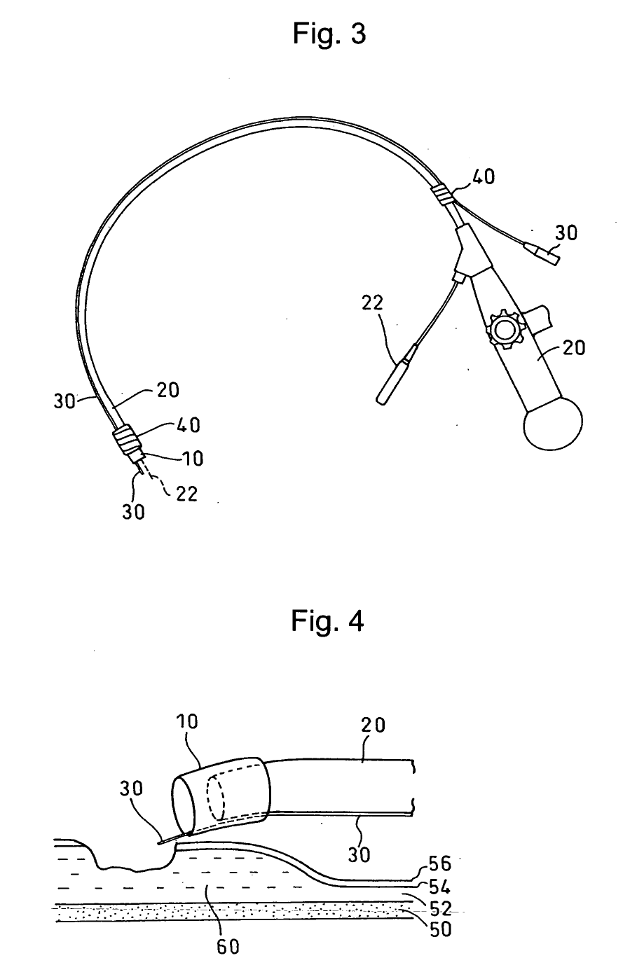

Among the treatment tools, necessary treatment tools are inserted in the insertion hole 14 as occasion may demand and then fixed to the hood 10 by winding, for example, a vinyl tape 40 as illustrated in FIG. 3. FIG. 3 shows a state where only the precutting knife 30 is fitted to the hood 10 of the endoscope 20 in which the grasping forceps 22 are inserted.

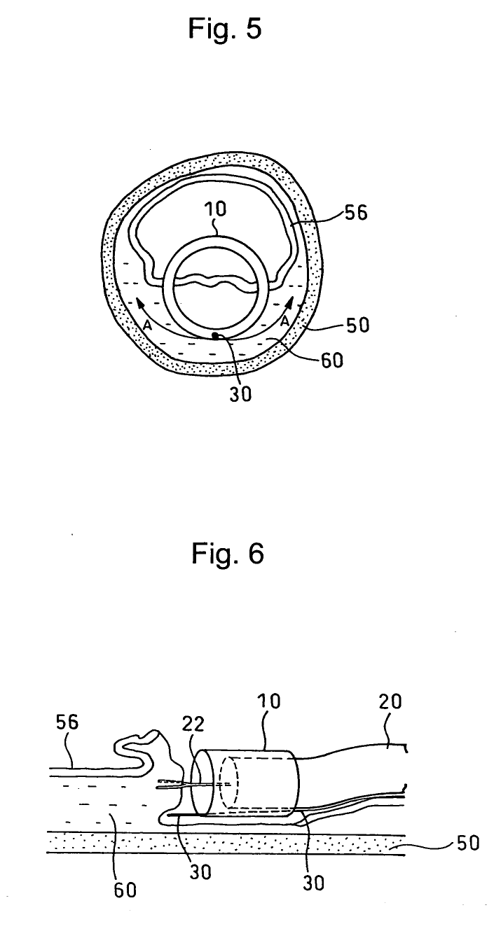

At the time of an operation, as shown in FIG. 4 for example, a physiological salt solution 60 is injected into a submucosal layer 52 between the muscularis propria 50 and muscularis mucosae 54, and then simple-cutting for dissecting a lesion from the mucosal epithelium 56 by the precutting knife 30, swing-cutting by rotating the precutting knife 30 as shown by the arrow A as shown in FIG. 5, and pull-swing cutting using both the precutting knife 30 and grasping forceps 22 as shown in FIG. 6, and so on can be performed. Further, avulsion can also be advanced by swing-cutting or pull-swing cutting while injecting the physiological salt solution at the deep submucosal layer.

In this first embodiment, the means for fitting the treatment tools are many insertion holes 14 extending in the axial direction of the tube 12, so that many treatment tools can be reliably fitted.

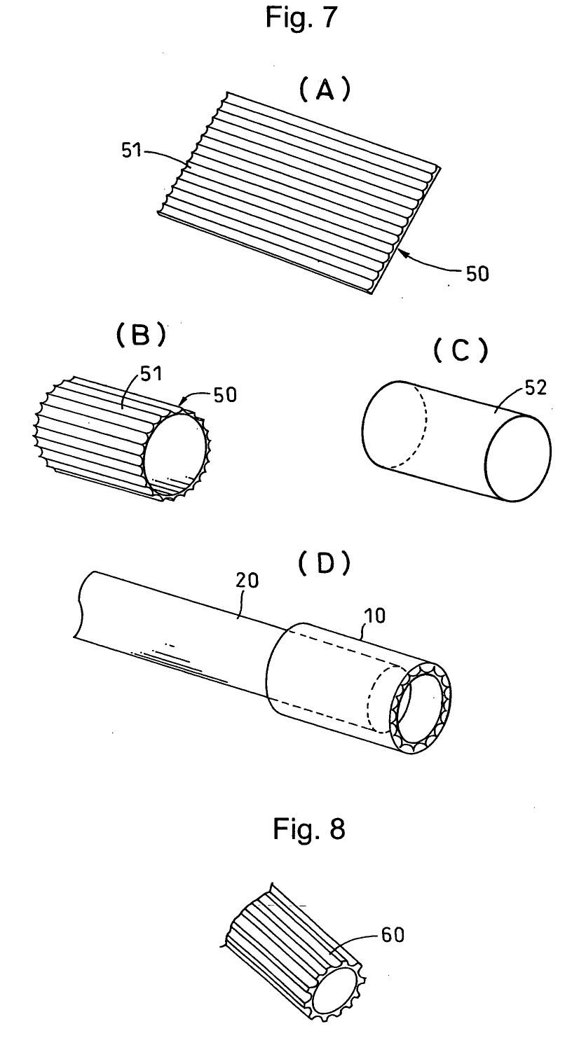

FIG. 7 show a second embodiment of the present invention.

The present embodiment is formed by rolling a silicon plate 50 having a number of convex lines 51 as shown in FIG. 7(A) so that the convex lines 51 extend in the axial direction on the outer side as shown in FIG. 7(B), and covering a thin silicon tube 52 thereon as shown in FIG. 7(C). The fitted state to an endoscope 20 is shown in FIG. 7(D).

According to the present embodiment, production is easy.

The sectional shape of the convex line 51 is not limited to the substantially triangular shape shown in FIG. 7, and may be substantially quadrilateral as in the case of the variation shown in FIG. 8. The treatment tool is fixed finally with a vinyl tape or the like, so that it may have slits 60 opened on the surface as shown in FIG. 8.

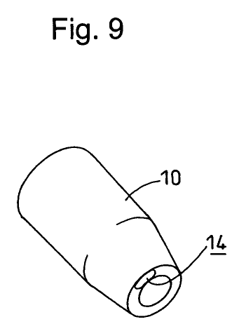

Various variations of the insertion holes 14 and the slits 60 are shown in FIG. 9

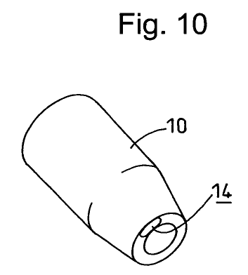

A variation in which the tip end of the hood 10 is tapered is shown in FIG. 10.

In the above-described embodiment, the material of the hood is a transparent silicon tube, so that the outer side of the hood can also be observed. The material of the hood is not limited to the transparent silicon tube, and the means for fixing the treatment tools is not limited to a vinyl tape, either.

The application object is not limited to superficial esophageal cancer, either, and the present invention is applicable to stomach cancers and colon cancers, etc.

### INDUSTRIAL APPLICABILITY

The present invention can be used for multi-purposes in various examinations, procedures, and treatments such as endoscopic ultrasonography (EUS), magnifying observation, and polypectomy, etc., in addition to EMR and EDS.

1. An endoscopic multi-purpose treatment hood in a tube shape in which a treatment tool fitting means for fitting treatment tools formed by a plurality of slits extending in the axial direction of a tube are formed on the periphery.

2. The endoscopic multi-purpose treatment hood according to claim 1, wherein the treatment tool fitting means is formed by providing a plurality of slits on the outer periphery of the tube.

3. The endoscopic multi-purpose treatment hood according to claim 1, wherein the treatment tool fitting means is formed by rolling a plate having a plurality of convex lines so that the convex lines extend in the axial direction on the outer side.

4. The endoscopic multi-purpose treatment hood according to claim 3, wherein the treatment tool fitting means is formed by covering a tube on the convex lines.

5. The endoscopic multi-purpose treatment hood according to claim 1, wherein the treatment tool fitting means is provided with a liquid injecting treatment tool.

6. An endoscope fitted with an endoscopic multi-purpose treatment hood in a tube shape in which a treatment tool fitting means for fitting treatment tools formed by a plurality of silts extending in the axial direction of a tube are formed on the periphery.

7. The endoscopic multi-purpose treatment hood according to claim 2, wherein the treatment tool fitting means is formed by covering a tube on the slits.
